(19) United States
(12) Patent Application Publication
(10) Pub. No.: US 2007/0107736 A1
(43) Pub. Date: May 17, 2007

(54) System and method for monitoring temperature while delivering anesthesia

(76) Inventor: Jan Karasek, Aurora, CO (US)

Correspondence Address: COOLEY GODWARD KRONISH LLP;ATTN: PATENT GROUP, Suite 500, 1200 - 19th Street, NW, WASHINGTON, DC, 20036-2402, US

(21) Appl. No.: 11/281,525

(22) Filed: Nov. 17, 2005

(51) Int. Cl. A61M 16/00; A62B 9/06

(52) U.S. Cl. 128/207.15; 128/207.14

(57) ABSTRACT

A system and method for orally monitoring a patient's core temperature while an artificial airway device is in the patient's airway is described. One embodiment includes a system for orally monitoring a patient's body temperature while delivering a gas into the patient's airway. The system comprises an artificial airway device, a thermometer affixed to the artificial airway device and a device connected to the thermometer that displays the temperature from the thermometer.

### FIELD OF THE INVENTION

The present invention relates generally to medical monitoring devices and artificial airway devices. In particular, but not by way of limitation, the present invention relates to devices for monitoring a patient's temperature.

### BACKGROUND OF THE INVENTION

Artificial airway devices, such as an laryngeal mask airway (“LMA”) or an endotracheal tube (“ETT”), have been utilized as a tool to support a patient's breathing and to deliver oxygen and other gases into a patient's airway. For example, during medical operations requiring anesthesia, an artificial airway device may be used to deliver anesthesia and other gases, such as oxygen or nitrous oxide, to the patient's lungs.

During these medical operations, it is very important to maintain the patient's body temperature. It has been found, for example, that hypothermia both increases the incidence of surgical infections and inhibits the patient's blood clotting systems, which makes the patient more likely to suffer increased intraoperative blood loss. As a consequence, the practice guidelines of the American Society of Anesthesiologists and the Joint Committee of Hospital Accreditation require that, throughout such medical operations, the patient's body temperature must be monitored.

Because the use of an artificial airway device creates certain difficulties for measuring a patient's temperature in or through the oral cavity, other sites such as the rectum may be used, resulting in less accurate measurement, increased stress, likelihood of injury and/or embarrassment for the patient. Moreover, widely available body surface temperature monitoring devices are not reliable because the body surface sensors are exposed to either the cold operating room environment (e.g., 60 to 65 degrees Fahrenheit) or to external heating blankets that are frequently used to warm the patient during surgery.

The importance of being able to accurately monitor a patient's temperature during surgery, and while the patient is under anesthesia, is without question. Although currently available esophageal temperature probes are able to accurately measure a patient's temperature, these probes include a relatively large plastic tube (e.g., approximately one-quarter of an inch in diameter and about fifteen inches long) that is inserted into the patients esophagus. As a consequence, the applicability of esophageal temperature probes during surgery is generally limited to instances where the patient is under complete general anesthesia and when the patient's artificial ventilation is provided by a breathing tube (e.g., an endotracheal tube) that is concurrently inserted into the patient's windpipe.

In the last decade, the insertion of a breathing tube all the way into the patient's windpipe became unnecessary due to the development of the Laryngeal Mask Airway (LMA). In use, the LMA does not extend into the patients windpipe, and as a consequence, the LMA provides less irritation than the endotracheal tube while maintaining sufficient “airway control.” Out of approximately 22 to 24 million surgical procedures performed each year in the U.S.A., it is estimated that up to 50% are now performed with an LMA instead of the “classical” windpipe intubation.

Unfortunately, the LMA completely occludes the entrance into the patient's throat; thus the insertion of the esophageal temperature probe is physically impossible. As a consequence, up to 12 million patients are deprived of the accurate temperature monitoring and their anesthesia providers must rely on surface probes that are subject to the previously described distortions. Accordingly, a system and method are needed to address the shortfalls of present technology and to provide other new and innovative features.

### SUMMARY OF THE INVENTION

To remedy the above described and other deficiencies of the current technology, an apparatus, system and method for orally monitoring a patient's temperature while an artificial airway device is in the patient's airway is disclosed. In one embodiment, the present invention includes an artificial airway device and a thermometer connected to the artificial airway device. Because the thermometer is connected to the artificial airway device, the device can be used to orally measure a patient's body temperature while delivering a gas into the patient's airway.

In another embodiment, the present invention encompasses a method for orally monitoring a patient's body temperature while an artificial airway device is within the patient's airway. In this embodiment an artificial airway device is first inserted, with a thermometer affixed thereto, into a patient's airway. Then, the thermometer is connected to a system capable of displaying a patient's body temperature. The thermometer then takes the patient's body temperature and the system displays the patient's body temperature.

In yet another embodiment, the present invention encompasses a system for orally monitoring a patient's body temperature while delivering a gas into the patient's airway. The system in this embodiment includes a thermometer affixed to an artificial airway device and a device connected to the thermometer that displays the temperature from the thermometer.

Accordingly, the present invention overcomes the problems with the present technology and addresses the needs of the patient care community. Exemplary embodiments of the present invention that are shown in the drawings are summarized below. These and other embodiments are more fully described in the Detailed Description section. It is to be understood, however, that there is no intention to limit the invention to the forms described in this Summary of the Invention or in the Detailed Description. One skilled in the art can recognize that there are numerous modifications, equivalents and alternative constructions that fall within the spirit and scope of the invention as expressed in the claims.

### BRIEF DESCRIPTION OF THE DRAWINGS

Various objects and advantages and a more complete understanding of the present invention are apparent and more readily appreciated by reference to the following Detailed Description and to the appended claims when taken in conjunction with the accompanying Drawings wherein:

FIG. 1 is a block diagram, which illustrates a system for orally monitoring a patient's body temperature in accordance with the present invention.

FIG. 2 is a flow chart of one method for orally monitoring a patient's body temperature in accordance with the present invention.

FIGS. 3 and 3A illustrate a schematic view and an exploded schematic view, respectively of a device according to an embodiment of the present invention.

FIGS. 4 and 4A illustrates a schematic view and an exploded schematic view, respectively of a device according to another embodiment of the present invention.

### DETAILED DESCRIPTION

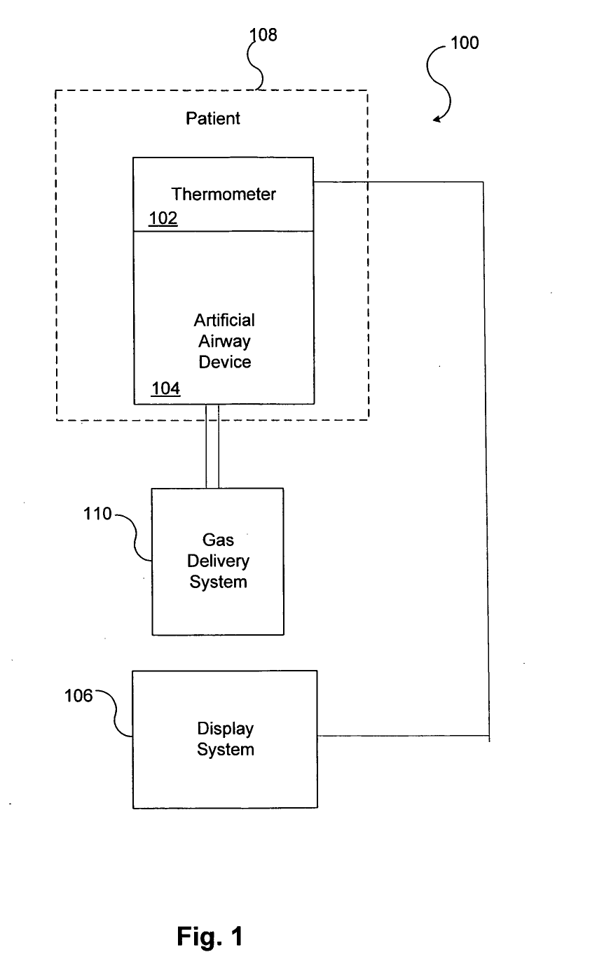

Referring now to the drawings, where like or similar elements are designated with identical reference numerals throughout the several views, and referring in particular to FIG. 1, it illustrates a system 100 for orally monitoring a patient's body temperature in accordance with the present invention. In accordance with several embodiments, the system 100 enables the temperature of a patient 108 to be orally monitored while an artificial airway device is in the patient's airway. This is accomplished by connecting a thermometer 102 to, or integrating the thermometer 102 with, the artificial airway device 104.

In the exemplary embodiment depicted in FIG. 1, the thermometer 102 is connected to the artificial airway device 104 and to a display system 106. As shown, the connection between the thermometer 102 and the display system 106 is represented by a solid line, but the connection is certainly not limited to wire line connections. In some embodiments for example, the thermometer 102 is connected by a wireless connection with the display system 106. As illustrated in FIG. 1, it is contemplated that the system will be constructed and implemented such that the thermometer 102 and artificial airway device 104 can be inserted into a patient 108.

As shown in FIG. 1 the artificial airway device 104 is also connected to a gas delivery system 110. In the exemplary embodiment, the gas delivery system 110 is utilized to deliver anesthesia, oxygen or other gases while the thermometer 102 is within the patient's 108 airway. Although the gas delivery system 110 and display system 106 are depicted as separate devices in the present embodiment, in another embodiment (not shown) the gas delivery system 110 and the display system 106 are incorporated into a single device.

By way of example only, the artificial airway device 104 in some embodiments is realized as an endotracheal tube (ETT) and in other embodiments the artificial airway device is implemented as a laryngeal mask airway (LMA). The thermometer 102 may be realized as any of a group of digital thermometers, electric thermometers, wireless thermometers, infrared thermometers, thermoscan thermometers, temperature sensors, thermal sensors, contact thermometers, thermocouple thermometers, temperature transducers, thermistors, temperature probes, radiation thermometers/pyrometers, analog thermometers and thermal diodes.

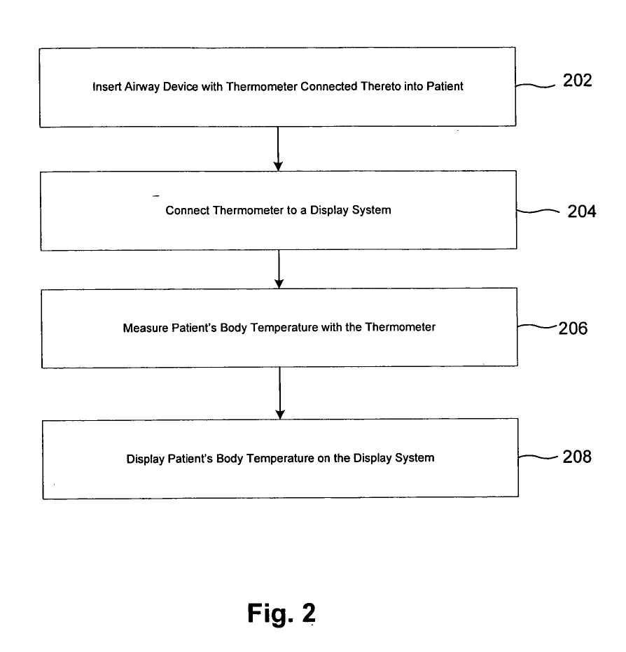

While referring to FIG. 1, simultaneous reference will be made to FIG. 2, which is a flowchart depicting steps carried out by the system 100 of FIG. 1 when orally monitoring a patient's body temperature in accordance with an exemplary embodiment of the present invention. As shown, the artificial airway device 104, with thermometer 102 connected thereto, is inserted into the patient 108 (Block 202), and once inserted, the thermometer 102 is connected to the display system 106 (Block 204). This may involve connecting a physical wire to the display system 106, or the thermometer 102 may be wirelessly connected. Next, the thermometer 102 measures the patient's 108 body temperature (Block 206), and the patient's 108 body temperature is then displayed on the display system 106 (Block 208). It should be recognized that the order of steps depicted in FIG. 2 may be varied without departing from the scope of the present invention. In another embodiment, for example, the thermometer 102 is connected to the display system 106 before the artificial airway device 104 (with thermometer 102 connected thereto) is inserted into the patient 108.

Although not depicted in FIG. 1 or 2, after the artificial airway device 104 is inserted into a patient's 108 airway, it can be connected to the gas delivery system 110. The exemplary embodiment of the present invention will therefore allow for the simultaneous delivery of gas, such as anesthesia or oxygen, into the patient 108 and the oral monitoring of a patient's body temperature with a single device. As used herein, oral or orally monitoring and oral or orally measuring are intended to encompass any monitoring or measuring that is done in or through the oral cavity of the patient, including but not limited to measuring or monitoring done at a patient's pharyngeal area and any other similar or surrounding area.

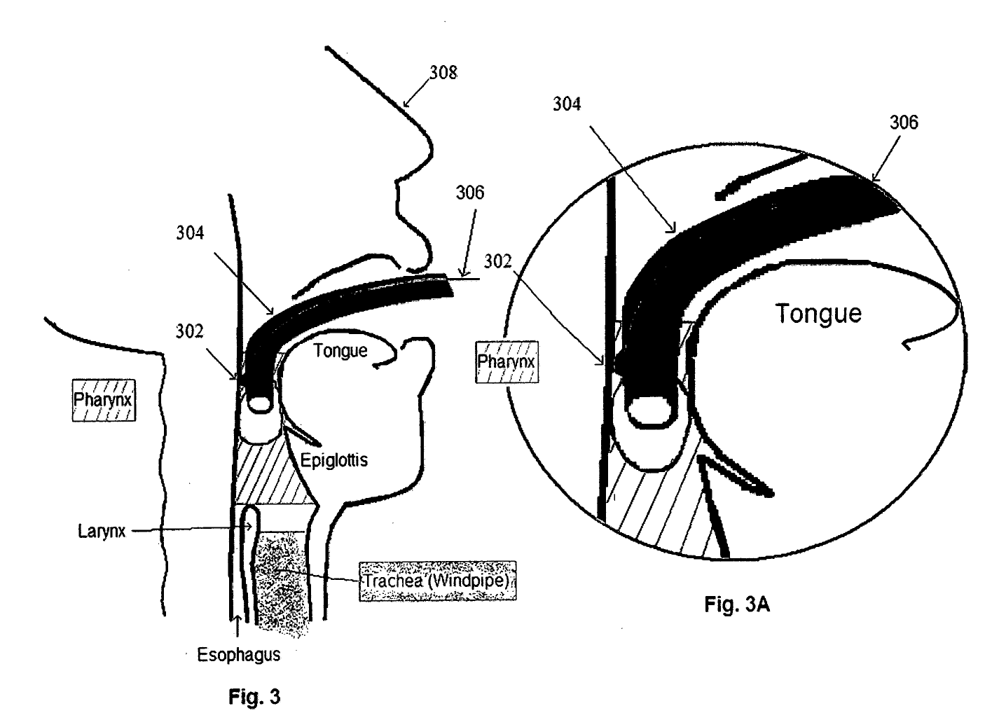

Referring next to FIG. 3, shown is a schematic view of a device where the artificial airway device 104 of FIG. 1 is realized as a laryngeal mask airway (LMA) 304. The LMA 304 in this embodiment, is depicted inside of a human patient 308 for purposes of example. In FIG. 3, and in an exploded view in FIG. 3A, the thermometer 302 is shown affixed to the LMA 304 at a location that allows the thermometer 302 to be in physical contact with the surface of the oropharyngx so as to enable the thermometer 302 to measure the patient's 308 core body temperature.

As depicted in FIG. 3, the LMA 304 is configured to be inserted into the patient's 308 throat, and the thermometer 302 is disposed relative to the LMA 304 so as to be capable of measuring a temperature at the patient's pharynx when the LMA 304 is inserted in the patient's throat. Given the relative proximity of an area like the pharynx to the patient's heart, if the patient's core body temperature changes, then such a change is prone to be quickly picked up by the thermometer 302, which makes a location like the pharynx a good location for measuring a patient's core body temperature. The core body temperature is considered by many to be to most appropriate temperature of a patient to measure due to the accuracy of the measurements and the insight, which the core body temperature provides into the patient's condition. As a consequence, several embodiments of the present invention provides a convenient and accurate measurement of arguably the most pertinent temperature of the patient—the core body temperature.

The thermometer 302 in the exemplary embodiment is also shown placed away from the tip of the LMA 304 to help make sure any temperature differential between the gases in the LMA 304 and the surrounding tissue does not affect the temperature read by the thermometer 302. For this reason the artificial airway device 104, 304, could also be insulated in order reduce or eliminate any errors caused by a temperature differential between the artificial airway device 104, 304, or the gases passing through it, and the part of the patient 308 the thermometer is measuring. The present embodiment is only exemplary, however, and it is contemplated that there are locations on the LMA 304 where the thermometer 302 may be placed.

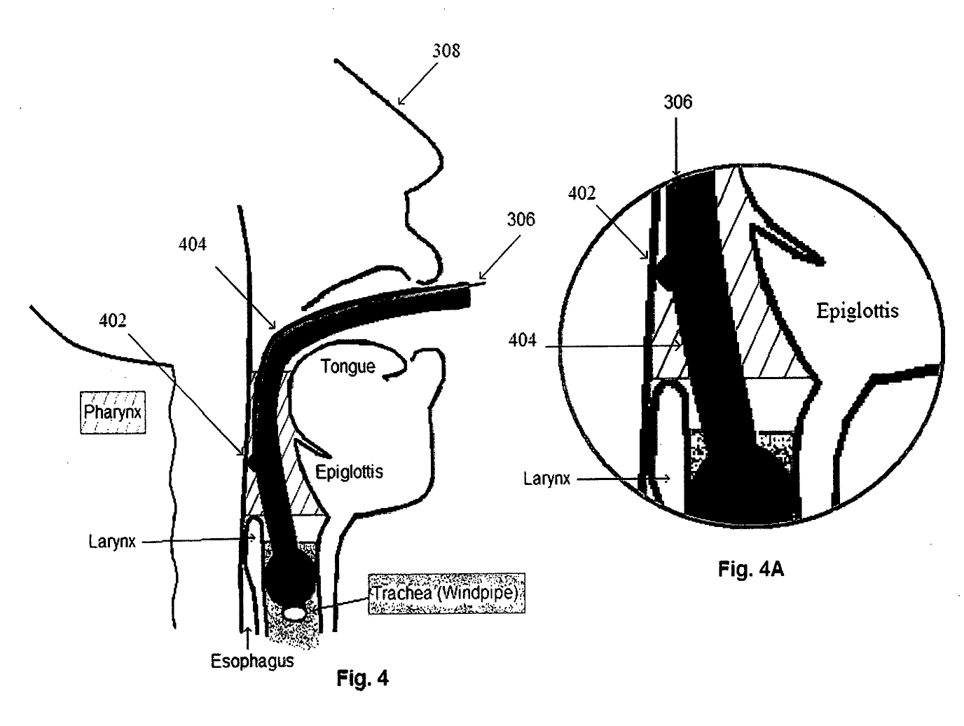

FIG. 3 also shows a wire 306 that runs from the thermometer through the inside of the body of the LMA. As discussed earlier, this wire 306 is only one potential, but not the only, means for connecting the thermometer 302 to a display system 106. The wire 306 may be imbedded in the structure of the LMA 304, inside or outside of the LMA 304, or there may be no wire at all, but one skilled in the art will be aware of other alternative embodiments that are well within the scope of the present invention. Furthermore, although FIGS. 3 and 4 depict a human patient 308, this is done for purposes of explanation and description, there is nothing that should suggest the present invention is limited to human patients or artificial airway devices used on human patients and it is contemplated that the present invention is applicable in other embodiments to non-human patients.

Referring next to FIG. 4, shown is a schematic diagram depicting a device according to another embodiment of the present invention. As shown in FIG. 4, the artificial airway device 104 of FIG. 1 is realized as an endotracheal tube (ETT) 404. In this embodiment, the thermometer 402 is located at a location where it can contact part of the patient's 308 pharynx. Although the thermometer 402 is located at the lower part of the pharynx in FIG. 4, this is just by way of example and not a limitation to the present invention.

In conclusion, the present invention provides, among other things, a system and method for orally monitoring a patient's temperature while an artificial airway device is in the patient's airway. Those skilled in the art can readily recognize that numerous variations and substitutions may be made in the invention, its use and its configuration to achieve substantially the same results as achieved by the embodiments described herein. Accordingly, there is no intention to limit the invention to the disclosed exemplary forms. Many variations, modifications and alternative constructions fall within the scope and spirit of the disclosed invention as expressed in the claims.

What is claimed is:

1. An apparatus for orally measuring a patient's body temperature while delivering a gas into the patient's airway, comprising:
an artificial airway device; and
a thermometer connected to the artificial airway device so as to enable a temperature of the patient to be measured while the artificial airway device is delivering the gas into the patient's airway.

2. The apparatus of claim 1 wherein the artificial airway device comprises a laryngeal mask airway (LMA).

3. The apparatus of claim 2 wherein the thermometer is connected to the laryngeal mask airway (LMA) so as to allow the thermometer to measure temperature at a pharynx of the patient while the LMA is in the patient's throat.

4. The apparatus of claim 1 wherein the artificial airway device comprises an endotracheal tube (ETT).

5. The apparatus of claim 4 wherein the thermometer is connected to the endotracheal tube (ETT) so as to allow the thermometer to measure temperature at a pharynx of the patient while the ETT is in the patient's windpipe.

6. The apparatus from claim 1 wherein the thermometer is selected from a group consisting of digital thermometers, electric thermometers, wireless thermometers, infrared thermometers, thermoscan thermometers, temperature sensors, thermal sensors, contact thermometers, thermocouple thermometers, temperature transducers, thermistors, temperature probes, radiation thermometers/pyrometers, analog thermometers, and thermal diodes.

7. The apparatus from claim 1 wherein the gas is a gas selected from a group consisting of anesthesia, oxygen and air.

8. The apparatus from claim 1 wherein the thermometer is insulated from a temperature inside of the artificial airway device.

9. The apparatus from claim 1 wherein the thermometer is integrated within the artificial airway device.

10. A method for orally monitoring a patient's body temperature comprising:
inserting the artificial airway device, with a thermometer affixed thereto, into the patient's airway;
connecting the thermometer to a system capable of displaying the patient's body temperature;
measuring the patient's body temperature using the thermometer; and
displaying the patient's body temperature.

11. The method of claim 10 further comprising of:
connecting the artificial airway device to a gas delivery system; and
delivering a gas to the patient.

12. The method from claim 10 wherein the thermometer is integrated within the artificial airway device.

13. An artificial airway device for measuring a patient's body temperature comprising:
a tubular structure with a first end and a second end, the tubular structure being configured for placement in a patient's airway, wherein the first end is configured to connect to a system for delivering gas into the tubular structure, and wherein the second end is open to allow the gas to pass from the tubular structure to the patient's airway; and
a thermometer that is connected to the tubular structure so as to allow for measurement of the patient's core body temperature when the tubular structure is within the airway of the patient.

14. The artificial airway device from claim 13 wherein the tubular structure is an integrated part of a laryngeal mask airway (LMA).

15. The artificial airway device from claim 14 wherein the thermometer is connected to the laryngeal mask airway (LMA) in such a way as to allow the thermometer to measure temperature at a pharynx of the patient while the LMA is in the patient's airway.

16. The artificial airway device from claim 13 wherein the tubular structure is an integrated part of an endotracheal tube (ETT).

17. The artificial airway device from claim 16 wherein the thermometer is connected to the endotracheal tube (ETT) in such a way as to allow the thermometer to measure temperature at a pharynx of the patient while the ETT is in the patient's airway.

18. The artificial airway device from claim 13 wherein the thermometer is selected from a group consisting of digital thermometers, electric thermometers, wireless thermometers, infrared thermometers, thermoscan thermometers, temperature sensors, thermal sensors, contact thermometers, thermocouple thermometers, temperature transducers, thermistors, temperature probes, radiation thermometers/pyrometers, analog thermometers, and thermal diodes.

19. The artificial airway device from claim 13 wherein the thermometer is integrated within the artificial airway device.

20. A system for orally monitoring a patient's body temperature while delivering a gas into the patient's airway, comprising:
an artificial airway device;
a thermometer affixed to the artificial airway device; and
a device connected to the thermometer that displays the temperature from the thermometer.

21. The system from claim 20 wherein the artificial airway device is connected to a gas delivery system.

22. The system from claim 21 wherein the gas delivery system is delivering a gas selected from a group of gases consisting of anesthesia, oxygen, nitrous oxide and air.

23. The system from claim 20 wherein the thermometer is selected from a group consisting of digital thermometers, electric thermometers, wireless thermometers, infrared thermometers, thermoscan thermometers, temperature sensors, thermal sensors, contact thermometers, thermocouple thermometers, temperature transducers, thermistors, temperature probes, radiation thermometers/pyrometers, analog thermometers, and thermal diodes.

24. The system from claim 20 wherein the thermometer is integrated within the artificial airway device.
